(19) United States
(12) Patent Application Publication
(10) Pub. No.: US 2025/0025671 A1
(43) Pub. Date: Jan. 23, 2025

(54) Catheter System

(71) Applicant: TERUMO KABUSHIKI KAISHA, Tokyo (JP)

(72) Inventors: Chisato IWAKIRI, Kanagawa (JP); Naoya SHIMADA, Kanagawa (JP)

(73) Assignee: TERUMO KABUSHIKI KAISHA, Tokyo (JP)

(21) Appl. No.: 18/775,735

(22) Filed: Jul. 17, 2024

(30) Foreign Application Priority Data
Jul. 19, 2023 (JP) 2023-117510

(51) Int. Cl. A61M 25/10; A61M 25/00

(52) U.S. Cl. CPC A61M 25/1006; A61M 25/0097; A61M 2025/0004; A61M 2025/0008; A61M 2025/1086; A61M 2025/109

(57) ABSTRACT

A catheter system configured to be inserted into a lumen of a living body includes: a flexible outer tube; an inner tube disposed in an inner cavity of the outer tube; a balloon having a tubular shape that connects a distal portion of the outer tube and a distal portion of the inner tube to each other, the balloon being configured to inflate radially inward with respect to the outer tube to allow insertion of a rod-like member through the inner tube, wherein the balloon has an outer peripheral portion that touches inner periphery of the lumen when the balloon protrudes in the distal direction from the distal end of the outer tube; and a removing unit, which is (i) a protrusion or recess formed at the outer peripheral portion of the balloon, or (ii) a part of the rod-like member inserted into the balloon.

### CROSS-REFERENCE TO RELATED APPLICATIONS

This application claims priority to Japanese Patent Application No. 2023-117510, filed on Jul. 19, 2023. The entire contents of these applications are incorporated herein by reference.

### BACKGROUND

The present disclosure relates to a catheter system.

JP 3921112 B2 discloses a balloon catheter for treating a lesion (constriction or obstruction) in the fallopian tube. This balloon catheter includes a flexible outer tube, an inner tube disposed in an inner cavity of the outer tube and movable in the axial direction relative to the outer tube, a tubular balloon that connects a distal portion of the outer tube and a distal portion of the inner tube to each other, and an operation unit disposed in a part of the outer tube. An endoscope is inserted into the balloon. When a user operates the operation unit, the endoscope travels along the outer tube in the distal direction.

### SUMMARY

It is desired to efficiently remove tissue in a lesion (such as fallopian tube cancer and constriction or obstruction) in a lumen of a living body.

(1) An aspect of the present invention is directed to a catheter system that is inserted into a lumen of a living body and capable of traveling along the lumen, the catheter system including: a flexible outer tube; an inner tube disposed in an inner cavity of the outer tube, being movable in an axial direction of the outer tube; a balloon having a tubular shape that connects a distal portion of the outer tube and a distal portion of the inner tube to each other and inflates radially inward with respect to the outer tube to allow insertion of a rod-like member through the inner tube; and a removing unit protruding in a distal direction from a distal end of the outer tube and inserted into the lumen of the living body to remove tissue in the lumen, wherein the balloon has an outer peripheral portion that touches inner periphery of the lumen when the balloon protrudes in the distal direction from the distal end of the outer tube, and the removing unit is a protrusion formed in the outer peripheral portion of the balloon or a recess formed in the outer peripheral portion or a part of the rod-like member inserted into the balloon.

According to this catheter system, when the balloon is allowed to travel into the lumen of the living body along the lumen, it is possible to effectively remove the tissue in the lumen by the removing unit, that is, the protrusion formed in the outer peripheral portion of the balloon, the recess formed in the outer peripheral portion, or a part of the rod-like member inserted into the balloon.

(2) In the catheter system according to (1), the removing unit may be the protrusion or the recess formed in the outer peripheral portion of the balloon.

With this configuration, the balloon is provided with the removing unit which is the protrusion or the recess, thereby enabling removal of the tissue with the balloon and also enabling a simple structure and reduction in manufacturing cost as compared with a configuration in which the removing unit is designed as a separate member. Providing the balloon with the protrusion or the recess easily achieves the removing unit.

(3) In the catheter system according to (2), the inner tube may have a proximal end provided with an inner tube hub having a port capable of supplying and discharging a perfusate, and the port may be communicated with an inner part of the inner tube and an inner part of the balloon.

Accordingly, when the tissue is removed by the removing unit of the balloon in a state in which the perfusate is supplied to the balloon through the port, discharging the perfusate from the port makes it possible to effectively collect the tissue from the port together with the perfusate.

(4) In the catheter system according to (1), the removing unit may be disposed in a distal portion of the rod-like member.

With this configuration, the distal portion of the rod-like member is provided with the removing unit, enabling removal of tissue in the lumen more and reliably.

(5) In the catheter system according to (1) or (4), the inner tube may have a first scale mark disposed at a regular interval in the axial direction, and the rod-like member may have a second scale mark that is disposed at a regular interval in the axial direction from a distal end toward a proximal end and is half the length of the interval of the first scale mark in the axial direction.

With this configuration, when inserting a distal end of the balloon into the lumen of the living body, the rod-like member is relatively moved in the proximal direction based on the second scale mark, and then, the inner tube is moved in the distal direction based on the first scale mark, thereby preventing the rod-like member from protruding in the distal direction from the balloon before the distal end of the balloon reaches a target position in the lumen.

(6) In the catheter system according to any one of (1) to (3), the rod-like member may be an endoscope.

Accordingly, it is possible to move the balloon forward into the lumen of the living body by checking, for example, an endoscopic image instead of checking the scale during operation. (Furthermore, labeling a lesion with a cell surface marker or the like may make it possible to check the lesion with an endoscopic image.)

According to the present invention, a catheter system includes a removing unit protruding from a distal end of an outer tube in a distal direction and inserted into a lumen of a living body to remove tissue in the lumen, and the removing unit is a protrusion or a recess formed in an outer peripheral portion of a balloon or a part of a rod-like member inserted into the balloon. Accordingly, when the balloon travels into the lumen of the living body, it is possible to remove the tissue in the lumen effectively by the removing unit.

### BRIEF DESCRIPTION OF DRAWINGS

FIG. 1 is a schematic view illustrating a configuration of a catheter system according to a first embodiment of the present invention;

FIG. 2 is a partially omitted cross-sectional view of the catheter system illustrated in FIG. 1;

FIG. 3 is a first view for describing falloposcopic tuboplasty using the catheter system illustrated in FIG. 1;

FIG. 4 is a second view for describing the falloposcopic tuboplasty illustrated in FIG. 3;

FIG. 5 is a third view for describing the falloposcopic tuboplasty illustrated in FIG. 4;

FIG. 6 is a fourth view for describing the falloposcopic tuboplasty illustrated in FIG. 5;

FIG. 7 is a fifth view for describing the falloposcopic tuboplasty illustrated in FIG. 6;

FIG. 8 is a sixth view for describing the falloposcopic tuboplasty illustrated in FIG. 7;

FIG. 9 is a seventh view for describing the falloposcopic tuboplasty illustrated in FIG. 8;

FIG. 10 is a view for describing falloposcopic tuboplasty according to a first modification;

FIG. 11 is a view for describing falloposcopic tuboplasty according to a second modification;

FIG. 12 is a schematic view illustrating a configuration of a catheter system according to a second embodiment of the present invention;

FIG. 13 is a first view for describing endoscopic biopsy of the fallopian tube illustrated in FIG. 12;

FIG. 14 is a second view for describing the endoscopic biopsy of the fallopian tube illustrated in FIG. 13; and

FIG. 15A is an enlarged cross-sectional view illustrating a rod-like member according to a first modification, and FIG. 15B is an enlarged cross-sectional view illustrating a rod-like member according to a second modification.

### DETAILED DESCRIPTION

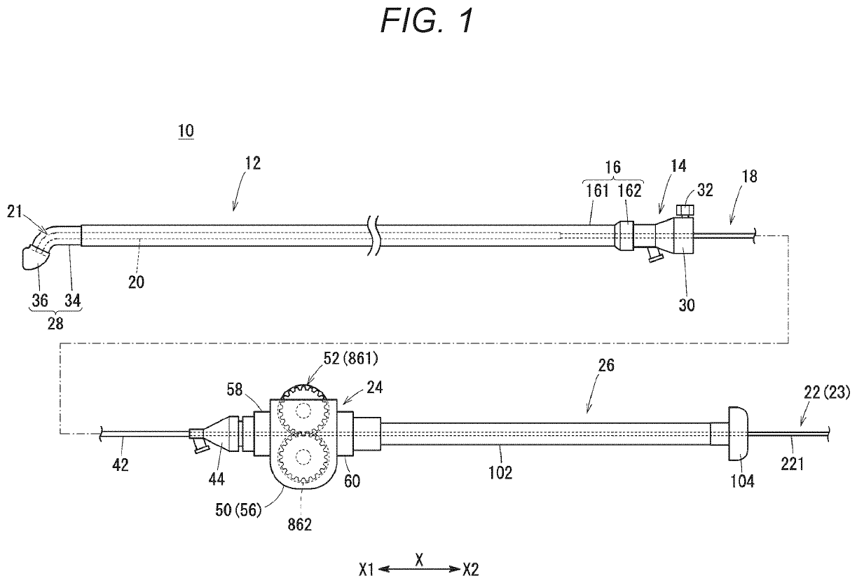

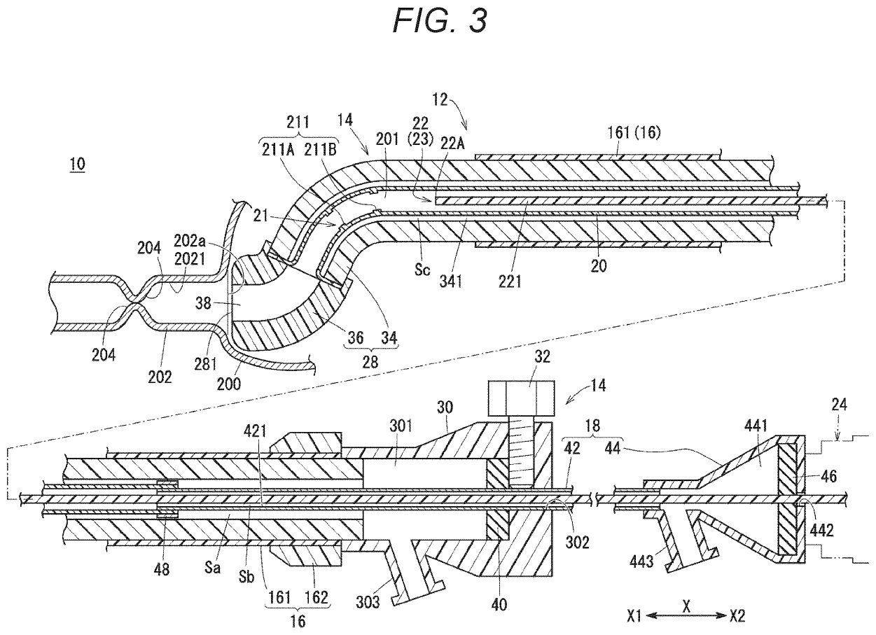

As illustrated in FIG. 1, a catheter system 10 according to a first embodiment is inserted into a lumen of a living body and travels along the lumen. The catheter system 10 is used, for example, in falloposcopic tuboplasty for treating a lesion 204 (lesion such as fallopian tube cancer and constriction or obstruction) in a fallopian tube 202, or a lumen (see FIG. 3). In addition to the fallopian tube 202, the catheter system 10 may be used for treating a lesion in a biological organ such as a blood vessel, a bile duct, a trachea, an esophagus, a urethra, and other organs. In the following description, the leftward direction (direction of arrow X1) of the catheter system 10 in FIG. 1 is referred to as the “distal direction”, and the rightward direction (direction of arrow X2) of the catheter system 10 in FIG. 1 is referred to as the “proximal direction”.

The catheter system 10 includes a catheter 12. The catheter 12 includes an outer tube 14, a slider 16, an inner tube 18, a balloon 20, an operation unit 24 for operating an endoscope 22 inserted into the inner tube 18, and a handle 26. That is to say, the catheter 12 is a balloon catheter. The endoscope 22 is a rod-like member 23 that is able to be inserted into the tubular balloon 20 through the inner tube 18.

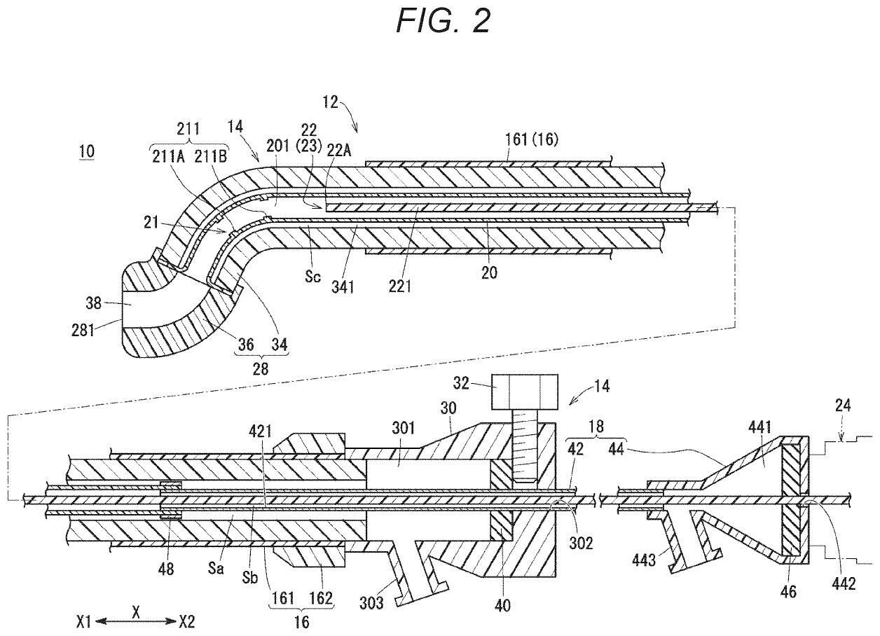

As illustrated in FIG. 2, the outer tube 14 has a flexible outer tube main body 28, an outer tube hub 30, and a fixing screw 32. The outer tube main body 28 includes a tubular body 34 and a distal member 36 (distal tip). Examples of the material for each of the tubular body 34 and the distal member 36 include polyolefins, polyesters, elastomer resins, flexible polymer materials, soft polyvinyl chloride, polyurethanes, polyamides, and fluororesins. The outer tube 14 is an outer catheter.

The tubular body 34 has a first inner cavity 341. The first inner cavity 341 penetrates the entire tubular body 34 in the axial direction. A distal portion of the tubular body 34 is curved in the axial direction of the tubular body 34 and formed into an arc. The tubular body 34 has a substantially constant outside diameter over the entire length in the axial direction.

The distal member 36 is disposed in the distal portion of the tubular body 34. The outer periphery of the distal member 36 is curved, corresponding to the shape of a uterine ostium 202a (see FIG. 3) curved relative to the travelling direction of the catheter 12. The distal member 36 has a balloon lead-out hole 38. The balloon lead-out hole 38 includes a distal opening 281 of the outer tube main body 28. The balloon 20 passes through the balloon lead-out hole 38 and is led out in the distal direction (direction of arrow X1) from the distal member 36.

The outer tube hub 30 is fixed to a proximal portion of the outer tube main body 28. The material for the outer tube hub 30 may be, for example, a hard resin or a metal material. Examples of the hard resin constituting the outer tube hub 30 include polycarbonates, acrylic resins, polyesters, polyolefins, styrene-based resins, polyamides, polysulfones, polyarylates, and polyetherimides. Examples of the metal material constituting the outer tube hub 30 include stainless steel, aluminum, and titanium.

The outer tube hub 30 is formed into a hollow shape. The outer tube hub 30 has a size that enables a user to operate easily. The outer tube hub 30 has a first space 301, a first insertion hole 302, and a first feeding port 303. The first space 301 is communicated with the first inner cavity 341 of the outer tube main body 28. The first insertion hole 302 is placed in the direction of arrow X2 relative to the first space 301. The inner tube 18 is inserted into the first insertion hole 302. The first feeding port 303 feeds an inflation fluid into the first space 301.

The inflation fluid causes the balloon 20 to inflate radially inward with respect to the outer tube main body 28. The inflation fluid is, for example, saline. The inflation fluid may be sterile water. The first space 301 of the outer tube hub 30 includes a first sealing member 40. The first sealing member 40 prevents the inflation fluid in the first space 301 from leaking from the first insertion hole 302 to the outside.

The fixing screw 32 is screwed into the outer tube hub 30. When the user tightens the fixing screw 32, the inner tube 18 is fixed to the outer tube hub 30. When the user loosens the fixing screw 32, the inner tube 18 becomes moveable relative to the outer tube hub 30.

The slider 16 is attached to the outer tube 14. The slider 16 slides on the outer periphery of the tubular body 34 in the axial direction of the outer tube main body 28. The entire length of the slider 16 in the axial direction is shorter than the entire length of the outer tube main body 28 in the axial direction. The slider 16 has a slider body 161 and a slider hub 162. The slider body 161 is a tubular member. The slider hub 162 is fixed to a proximal portion of the slider body 161. The slider hub 162 is formed into a tubular shape.

In a state in which the slider 16 is moved farthest in the direction of arrow X1 relative to the tubular body 34 (in an initial state of the slider 16), the distal portion of the tubular body 34 extends linearly, corresponding to the shape of the slider body 161. In contrast, when the slider 16 is moved in the direction of arrow X2 relative to the tubular body 34, the distal portion of the tubular body 34 is exposed from the slider 16 in the direction of arrow X1. At this time, the distal portion of the tubular body 34 is curved in an arc shape. Before the catheter system 10 is used, the catheter system 10 is stored with the distal portion of the tubular body 34 being curved.

The inner tube 18 includes an inner tube main body 42 and an inner tube hub 44. The material for the inner tube main body 42 may be, for example, a relatively hard resin material or a metal material. Examples of the hard resin constituting the inner tube main body 42 include polycarbonates, acrylic resins, polyesters, polyolefins, styrene-based resins, polyamides, polysulfones, polyarylates, and polyetherimides. Examples of the metal material constituting the inner tube main body 42 include stainless steel, aluminum, and titanium. The inner tube main body 42 has a second inner cavity 421 penetrating the entire inner tube main body 42 in the axial direction. The inner tube 18 is an inner catheter.

The inner tube main body 42 is disposed in the first inner cavity 341 of the outer tube main body 28, being movable in the axial direction of the outer tube main body 28. The inner tube main body 42 is inserted into the outer tube hub 30. A distal end of the inner tube main body 42 is disposed in the direction of arrow X2 relative to a distal end of the tubular body 34. Between the outer periphery of the inner tube main body 42 and the inner periphery of the tubular body 34, there is an outer lumen Sa through which the inflation fluid flows.

Into the second inner cavity 421 of the inner tube main body 42, an insertion section 221 of the endoscope 22 is inserted. In a state in which the insertion section 221 is inserted into the second inner cavity 421 of the inner tube main body 42, an inner lumen Sb through which a perfusate flows is disposed between the inner tube main body 42 and the insertion section 221.

The inner tube hub 44 is fixed to a proximal portion of the inner tube main body 42. The inner tube hub 44 is formed into a hollow shape. The inner tube hub 44 has a second space 441, a second insertion hole 442, and a second feeding port 443. The second space 441 is communicated with the second inner cavity 421 of the inner tube main body 42. The second insertion hole 442 is placed in the direction of arrow X2 relative to the second space 441. The insertion section 221 of the endoscope 22 is inserted into the second insertion hole 442. The second feeding port 443 feeds the perfusate into the second space 441.

The perfusate is, for example, saline. The second space 441 of the inner tube hub 44 is provided with a second sealing member 46. The second sealing member 46 prevents the perfusate in the second space 441 from leaking from the second insertion hole 442 to the outside.

The balloon 20 is a tubular member that connects a distal portion of the outer tube main body 28 and a distal portion of the inner tube main body 42 to each other. The balloon 20 is inflated by the inflation fluid radially inward with respect to the outer tube main body 28. In other words, the balloon 20 is elastically deformable in the radial direction. Examples of the material for the balloon 20 include polyolefins, polyesters, elastomer resins, flexible polymer materials, soft polyvinyl chloride, polyurethanes, polyamides, polyisoprenes, and fluororesins.

One end portion of the balloon 20 is bonded to the distal portion of the outer tube main body 28 with an adhesive (not illustrated). Alternatively, one end portion of the balloon 20 may be, for example, welded to the distal portion of the outer tube main body 28. One end portion of the balloon 20 is sandwiched between the distal end of the tubular body 34 and the distal member 36.

The other end portion of the balloon 20 is fixed to the outer periphery of the distal portion of the inner tube main body 42 by a balloon fixing member 48. Alternatively, the other end portion of the balloon 20 may be bonded to a distal portion of the inner periphery of the inner tube main body 42 with an adhesive. The other end portion of the balloon 20 may be welded to the distal portion of the inner periphery of the inner tube main body 42.

The balloon 20 has an inner cavity 201 into which the insertion section 221 of the endoscope 22 is allowed to insert. A bag-shaped outer space Sc having a closed distal end is disposed between the outer periphery of the balloon 20 and the inner periphery of the tubular body 34.

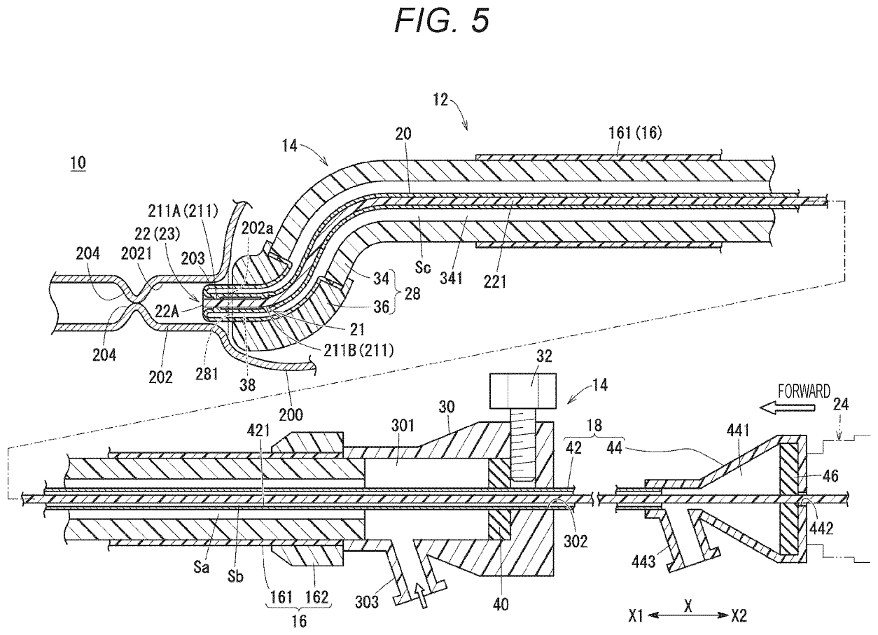

With the balloon 20 inflated radially inward, a pushing force (pushing force in the distal direction) is transferred from the inner tube main body 42 to the balloon 20, whereby the balloon 20 protrudes in the distal direction from the distal opening 281 of the outer tube main body 28 (see FIG. 5). At this time, a distal portion of the balloon 20 is turned inside out so that the inner surface of the balloon 20 faces outward. In other words, a portion of the balloon 20 protruding in the direction of arrow X1 from the distal opening 281 of the outer tube main body 28 includes a portion where the wall of the balloon 20 is double folded in the radial direction.

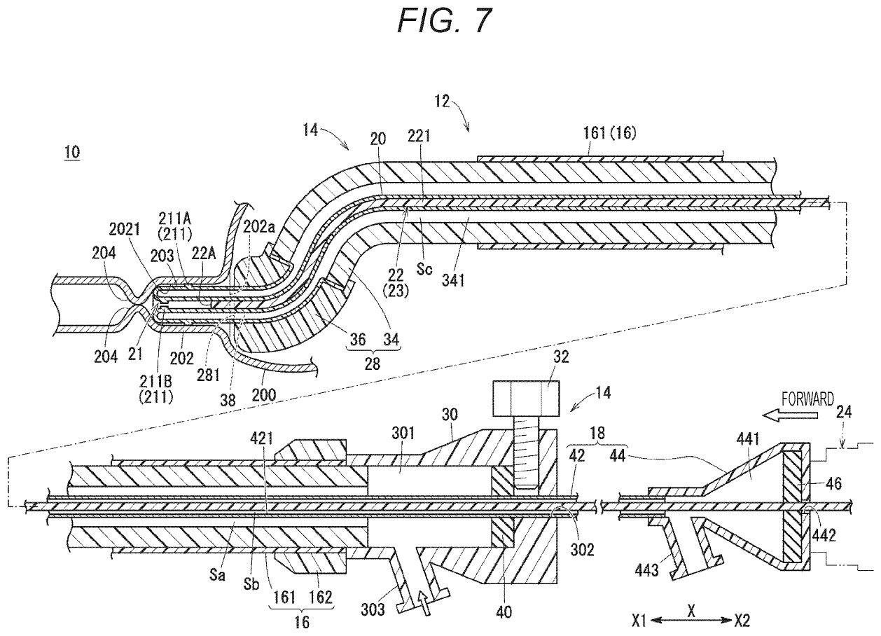

As illustrated in FIG. 7, the balloon 20 has an outer peripheral portion 203 disposed on the outer periphery side of the balloon 20 when the balloon 20 protrudes in the distal direction from a distal end of the outer tube 14. The outer peripheral portion 203 touches inner periphery 2021 of the fallopian tube 202 of the living body. In a state in which the balloon 20 is housed in the outer tube 14, the outer peripheral portion 203 is disposed on the inner periphery side.

The outer peripheral portion 203 of the balloon 20 includes a removing unit 21 capable of removing tissue in the living body. When the balloon 20 protrudes in the distal direction from the distal end of the outer tube 14, the outer peripheral portion 203 of the balloon 20 is inserted into the fallopian tube 202 of the living body and touches the inner periphery 2021 of the fallopian tube 202, whereby the removing unit 21 touches the inner periphery 2021 of the fallopian tube 202. The removing unit 21 is a protrusion 211 formed in the outer peripheral portion 203 of the balloon 20. The protrusion 211 protrudes radially outward from the outer peripheral portion 203. The protrusion 211 is formed into an annular shape along the circumferential direction of the balloon 20. As illustrated in FIG. 2, in a state in which the balloon 20 is housed in the outer tube 14, the protrusion 211 protrudes radially inward and faces the inner cavity 201.

Note that the protrusion 211 is not limited to an annular shape formed along the circumferential direction of the balloon 20. For example, the balloon 20 may be provided with a plurality of protrusions 211 spaced apart from each other in the circumferential direction of the balloon 20.

The protrusion 211 includes a first protruding part 211A and a second protruding part 211B. The first protruding part 211A and the second protruding part 211B are separated from each other in the axial direction (directions of arrow X) of the balloon 20. In a state in which the balloon 20 is housed in the outer tube 14, the first protruding part 211A is disposed in the distal direction (direction of arrow X1) relative to the second protruding part 211B. Note that each of the first protruding part 211A and the second protruding part 211B is not limitedly disposed singly. For example, the number of first and second protruding parts 211A and 211B may be two or more. The first protruding part 211A and the second protruding part 211B may have the same shape. Alternatively, the first protruding part 211A and the second protruding part 211B may have different shapes.

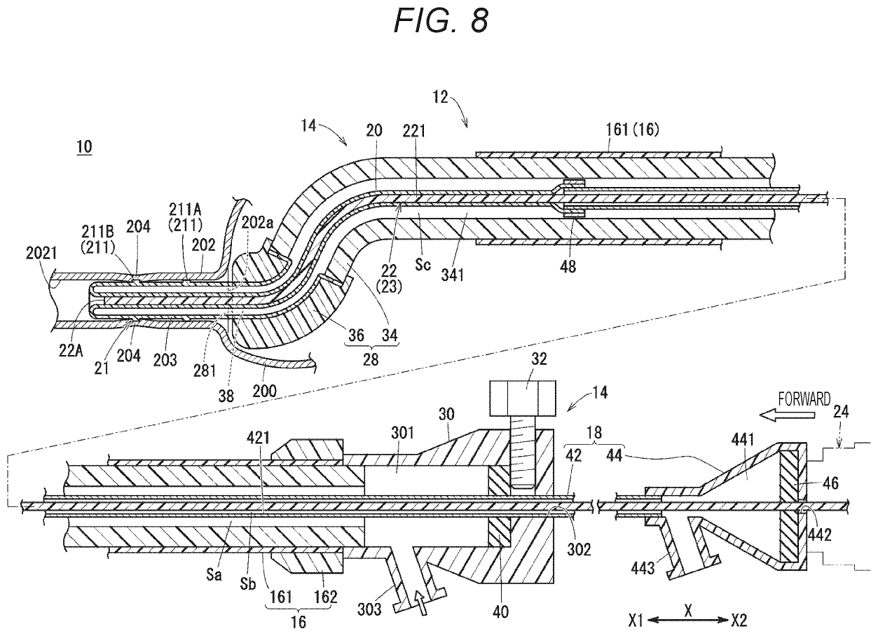

The first protruding part 211A is capable of removing tissue between the lesion 204 in the fallopian tube 202 and the uterine ostium 202a (see FIG. 7). The second protruding part 211B is capable of removing tissue in the lesion 204 in the fallopian tube 202 (see FIG. 8). Note that the protrusion 211 is not limited to the configuration including the first and second protruding parts 211A and 211B. The protrusion 211 may include at least the second protruding part 211B capable of removing the tissue in the lesion 204.

The removing unit 21 is not limited to the protrusion 211 protruding radially outward from the outer peripheral portion 203 of the balloon 20. For example, the removing unit 21 may be a recess that is depressed radially inward from the outer peripheral portion 203 of the balloon 20.

As illustrated in FIG. 2, the endoscope 22 is used for observing the fallopian tube 202. The endoscope 22 includes the flexible insertion section 221. The insertion section 221 is inserted into the second inner cavity 421 of the inner tube main body 42 and the inner cavity 201 of the balloon 20.

The insertion section 221 includes a light guide (not illustrated), a lens unit, an image guide, and a sheath. The light guide guides light from a light source (not illustrated) connected to a proximal portion of the insertion section 221 toward a distal end of the insertion section 221. The lens unit is an objective lens placed at a distal portion of the insertion section 221. The image guide guides an image obtained by the lens unit in the proximal direction of the insertion section 221. The sheath is a tube that protects the light guide, the lens unit, and the image guide. The endoscope 22 is used together with a display unit such as a display and an imaging control device for displaying a captured image (endoscopic image) on the display unit. Note that, in FIG. 2, the configuration of the insertion section 221 is illustrated in a simple manner.

As illustrated in FIG. 1, the operation unit 24 is disposed outside a proximal end of the inner tube main body 42. The operation unit 24 moves the insertion section 221 of the endoscope 22 in the axial direction (directions of arrow X) of the catheter 12 relative to the inner tube 18. In other words, the operation unit 24 causes the endoscope 22 inserted into the inner cavity 201 of the balloon 20 through the inner tube main body 42 to move forward and backward along the inner tube main body 42.

The operation unit 24 has a case 50 and a delivery member 52. The case 50 has a case body 56, a first connection 58, and a second connection 60. The first connection 58 protrudes from the case body 56 in the distal direction (direction of arrow X1). A proximal end of the inner tube hub 44 is connected to the first connection 58. The second connection 60 protrudes from the case 50 in the proximal direction (direction of arrow X2). The handle 26 is fixed to the second connection 60.

The delivery member 52 is disposed inside the case 50. The delivery member 52 includes first and second rotators 861 and 862. The first rotator 861 and the second rotator 862 are disposed side by side in the vertical direction of the case body 56. The first and second rotators 861 and 862 are supported in a rotatable manner relative to the case 50. The insertion section 221 of the endoscope 22 is sandwiched between the first rotator 861 and the second rotator 862. Rotation of the first and second rotators 861 and 862 delivers the endoscope 22 toward the inner tube main body 42.

The handle 26 includes a tubular member 102 and a protective tube 104. The tubular member 102 is formed into a tubular shape. A distal end of the tubular member 102 is connected to a proximal end of the second connection 60 in the operation unit 24. The protective tube 104 is fixed to a proximal end of the tubular member 102.

Hereinafter described is falloposcopic tuboplasty using the catheter system 10.

The falloposcopic tuboplasty involves preparation step to prepare the catheter system 10. During the preparation step, with the inner tube main body 42 completely drawn from the outer tube main body 28 in the direction of arrow X2, the fixing screw 32 is tightened. Accordingly, the inner tube main body 42 is fixed to the outer tube main body 28. The slider 16 is brought to the initial state, whereby the slider body 161 causes a straight stretch of the distal portion of the tubular body 34.

Subsequently, in insertion step, the catheter 12 is inserted transcervically to uterine fundus 200. As illustrated in FIG. 3, the slider 16 is pulled back relative to the outer tube main body 28 in the proximal direction (direction of arrow X2) of the outer tube main body 28. Accordingly, the distal portion of the tubular body 34 is exposed from the slider 16 and curved. At this time, the distal opening 281 of the outer tube main body 28 is placed near the uterine ostium 202a.

In the insertion step, the endoscope 22 is inserted into the second inner cavity 421 of the inner tube main body 42 through the protective tube 104 of the handle 26. After a distal portion 22A of the endoscope 22 is inserted into the tubular member 102 of the handle 26, a user allows the endoscope 22 to travel in the distal direction (direction of arrow X1). The distal portion 22A of the endoscope 22 is inserted into the operation unit 24 from the second connection 60.

As illustrated in FIG. 1, rotating the first rotator 861 of the operation unit 24 clockwise causes the second rotator 862 to rotate counterclockwise, or oppositely from the first rotator 861. Along with the rotation of the first and second rotators 861 and 862, the endoscope 22 is delivered in the distal direction (travelling direction, direction of arrow X1).

As illustrated in FIG. 3, in the insertion step, the distal portion 22A of the endoscope 22 travels into the second inner cavity 421 of the inner tube main body 42 through the operation unit 24 (see FIG. 1). A further operation of the first rotator 861 of the operation unit 24 causes the endoscope 22 to move forward along the second inner cavity 421 of the inner tube main body 42 and the distal portion 22A of the endoscope 22 to move forward up to the distal portion of the balloon 20. At this time, the protrusion 211 of the removing unit 21 is disposed inside the balloon 20 and faces the inner cavity 201.

After that, in check step, the user moves the endoscope 22 forward to place a distal end of the endoscope 22 at a distal end of the outer tube main body 28 (distal opening of the balloon lead-out hole 38), thereby checking the uterine ostium 202a in an image captured by the endoscope 22. Next, the fixing screw 32 is loosened to release the inner tube main body 42 fastened and fixed to the outer tube 14.

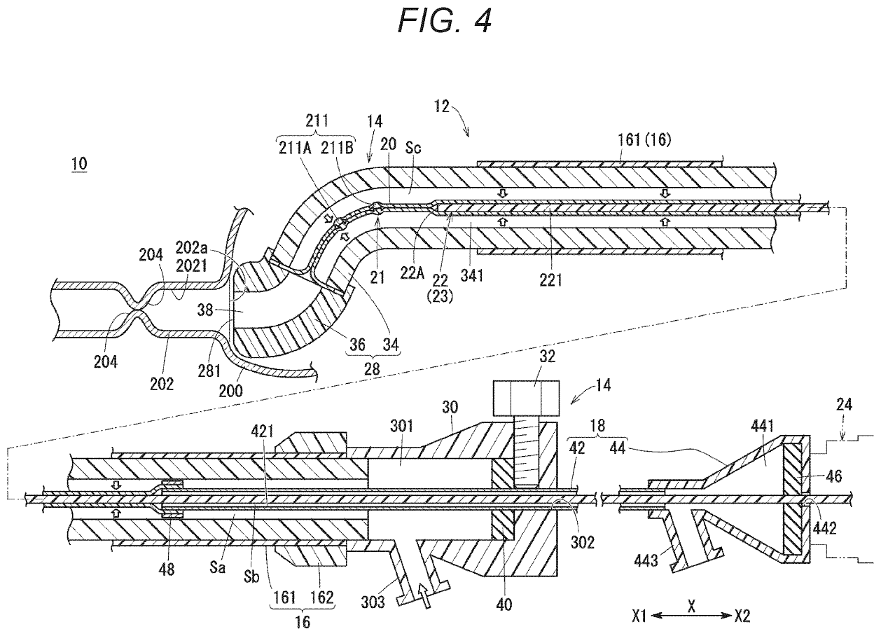

The next step is balloon lead-out step. As illustrated in FIG. 4, in pressurization step during the balloon lead-out step, the inflation fluid is supplied to the first feeding port 303 for pressurization. The inflation fluid is supplied through the outer lumen Sa from the first feeding port 303 to the outer space Sc of the balloon 20 between the outer tube main body 28 and the inner tube main body 42 in the inner cavity of the outer tube main body 28. In the pressurization step, the outer space Sc is pressurized with the inflation fluid at 5 to 9 atm. Note that the outer space Sc may be pressurized with the inflation fluid at 5 to 9 atm after the endoscope 22 is temporarily moved backward relative to the outer tube 14. It is further preferable that the outer space Sc is pressurized at 6 atm.

The balloon 20 is pressed radially inward by the inflation fluid supplied to the outer space Sc and elastically deformed. In other words, a portion of the balloon 20 placed on the radially outer side of the insertion section 221 is in close contact with the outer periphery of the insertion section 221. Portions of the balloon 20 placed in the direction of arrow X1 relative to the distal end of the insertion section 221 touch each other by inner surfaces.

After that, the user loosens the fixing screw 32 and operates the inner tube hub 44 in a state in which the outer space Sc is pressurized by the inflation fluid, thereby allowing the inner tube main body 42 to move forward relative to the outer tube main body 28 (forward movement step). In this forward movement step, the outer space Sc is continuously pressurized at 5 to 9 atm by the inflation fluid. Accordingly, as illustrated in FIG. 5, the balloon 20 pushed in the distal direction (direction of arrow X1) by the inner tube main body 42 moves forward relative to the outer tube main body 28. In other words, when a pushing force is transferred from the inner tube main body 42 to the balloon 20, the balloon 20 protrudes in the direction of arrow X1 from the distal opening 281 of the outer tube main body 28 together with the insertion section 221. Along with the forward movement of the balloon 20, the removing unit 21 (protrusion 211) moves in the distal direction along the inner cavity 201.

In the forward movement step, one end portion of the balloon 20 is fixed to the distal portion of the outer tube main body 28. Therefore, when the balloon 20 moves forward, the distal portion (protruding portion) of the balloon 20 turns inside out. In other words, the inner surface of the balloon 20 faces outward at the distal portion of the balloon 20. The portion of the balloon 20 facing outward is the outer peripheral portion 203.

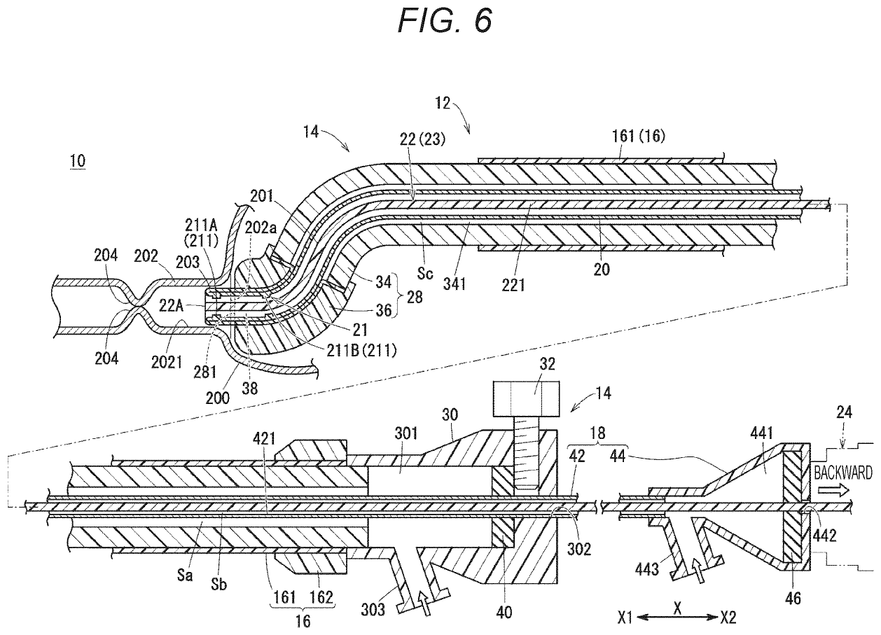

Subsequently, the user determines whether the endoscope 22 has reached the distal end of the balloon 20 based on the endoscopic image. When the balloon 20 is placed in front of the lesion 204, as illustrated in FIG. 6, the perfusate is supplied to the second feeding port 443 (supply step). Accordingly, the perfusate flows between the balloon 20 and the insertion section 221 of the endoscope 22 through the inner lumen Sb. The perfusate separates the balloon 20 from the insertion section 221 radially outward. In the perfusate supply step, the perfusate is supplied while the outer space Sc of the balloon 20 is continuously pressurized at 1 to 7 atm.

Next, the user moves the endoscope 22 backward by a predetermined distance (backward movement step). In the backward movement step, when the user rotates the first rotator 861 of the operation unit 24 counterclockwise, the second rotator 862 rotates clockwise. The first and second rotators 861 and 862 causes the insertion section 221 of the endoscope 22 to move backward in the proximal direction (direction of arrow X2). In the backward movement step, the endoscope 22 moves backward while the outer space Sc of the balloon 20 is continuously pressurized at 1 to 7 atm. After that, the pressurization step and the forward movement step are repeated.

In other words, in the falloposcopic tuboplasty using the catheter system 10, the traveling operation (forward movement) of the balloon 20 in the distal direction during the balloon lead-out step, the supply of the perfusate to a part between the inner tube main body 42 and the endoscope 22 during the supply step, and the backward movement of the endoscope 22 relative to the inner tube main body 42 and the balloon 20 during the backward movement step are all performed while the space is pressurized at 1 to 7 atm by the inflation fluid (perfusate).

As illustrated in FIG. 7, in the forward movement step, the distal portion of the balloon 20 moves forward and touches the lesion 204. The outer peripheral portion 203 of the balloon 20 touches the inner periphery 2021 of the fallopian tube 202. At this time, the first protruding part 211A of the removing unit 21 touches the inner periphery 2021 of the fallopian tube 202. The protrusion 211 slightly presses the inner periphery 2021 of the fallopian tube 202 radially outward. After that, continuing the forward movement step causes the outer peripheral portion 203 of the balloon 20 to move forward while touching the inner periphery 2021 of the fallopian tube 202. The first protruding part 211A of the removing unit 21 moves forward while touching the inner periphery 2021 of the fallopian tube 202. At this time, tissue in the inner periphery 2021 of the fallopian tube 202 comes into contact with the first protruding part 211A.

As illustrated in FIG. 8, when the balloon 20 thoroughly passes through the lesion 204, the lesion 204 is spread out by the outer peripheral portion 203 of the balloon 20. In other words, the constriction or obstruction of the fallopian tube 202 is improved. Along with the forward movement of the balloon 20, the second protruding part 211B of the removing unit 21 is placed at a position facing the lesion 204 and touches the inner periphery of the lesion 204 (fallopian tube 202). Along with the forward movement of the balloon 20, the second protruding part 211B moves forward while touching the inner periphery 2021, and the tissue in the inner periphery of the lesion 204 comes into contact with the second protruding part 211B. With the balloon 20 protruding in the distal direction from the distal end of the outer tube 14, the second protruding part 211B is disposed in the distal direction (direction of arrow X1) relative to the first protruding part 211A.

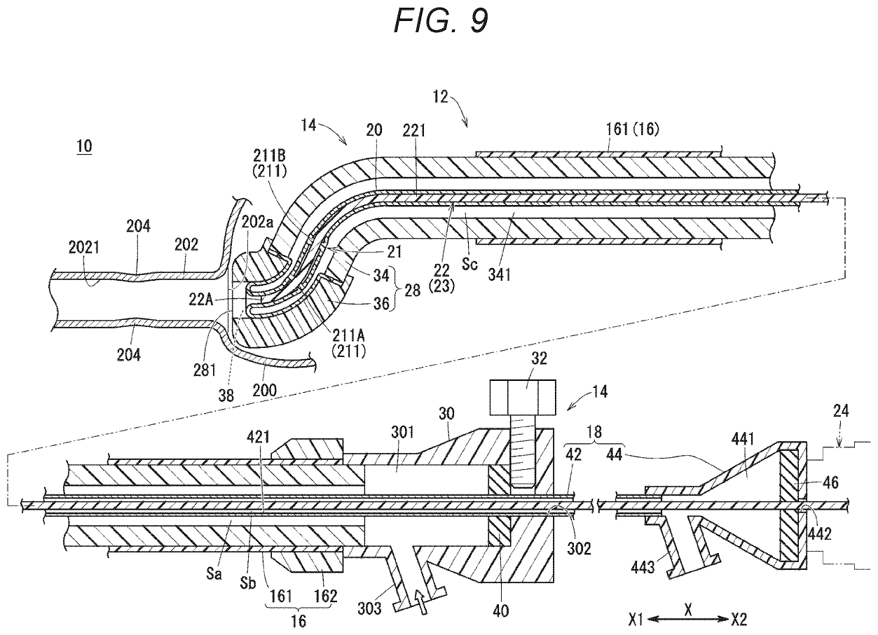

After the lesion 204 is expanded radially outward by the balloon 20, the catheter 12 and the endoscope 22 are extracted from the uterus (extraction step). As illustrated in FIG. 9, in the extraction step, the inner tube main body 42 may be pulled to move the balloon 20 backward while the perfusate is injected through the second feeding port 443, and simultaneously, the endoscope 22 may be operated to be placed at the distal portion of the balloon 20. Accordingly, it is possible to remove the catheter 12 and the endoscope 22 from the uterus while observing the inside of the fallopian tube 202 with the endoscope 22.

In the extraction step, along with the backward movement of the balloon 20, the outer peripheral portion 203 and the protrusion 211 of the balloon 20 move slidably in contact with the inner periphery 2021 of the fallopian tube 202, and the second protruding part 211B of the protrusion 211 scrapes and removes the tissue in the inner periphery of the lesion 204. The first protruding part 211A of the protrusion 211 scrapes and removes tissue in the inner periphery 2021 of the fallopian tube 202 which is placed in the proximal direction (direction of arrow X2) relative to the lesion 204. At this time, the tissue in the lesion 204 is caught on the second protruding part 211B protruding from the outer peripheral portion 203 and removed. The tissue placed in the proximal direction relative the lesion 204 is caught on the first protruding part 211A protruding from the outer peripheral portion 203 and removed. The removed tissue remains around the first protruding part 211A and the second protruding part 211B in the outer peripheral portion 203 of the balloon 20. On completion of the extraction step, the falloposcopic tuboplasty ends. After taking out the catheter system 10 from the living body, the balloon 20 is taken out from the outer tube 14, and the balloon 20 is expanded to collect the tissue in the living body removed by the removing unit 21. In a case in which the removing unit 21 is a recess disposed in the outer peripheral portion 203 of the balloon 20, the tissue in the inner periphery 2021 of the fallopian tube 202 can be caught and removed around the boundary between the recess and the outer peripheral portion 203.

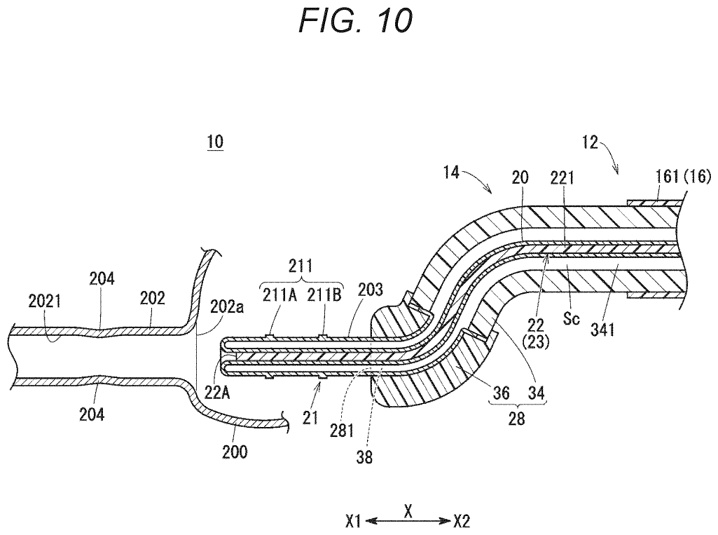

In the extraction step, the balloon 20 is not limitedly extracted from the uterine fundus 200 after moving backward relative the outer tube 14 and housed in the outer tube 14. For example, as illustrated in FIG. 10, without housing the balloon 20 in the outer tube 14, the catheter system 10 including the balloon 20 may be extracted from the uterine fundus 200 with the balloon 20 protruding in the distal direction (direction of arrow X1) from the distal end of the outer tube 14 and the removing unit 21 being exposed on the outer peripheral portion 203.

It is also possible to remove tissue around the lesion 204 prior to the treatment of the lesion 204 of the fallopian tube 202. In this case, as illustrated in FIG. 7, during the forward movement step of the catheter system 10, after the outer peripheral portion 203 of the balloon 20 touches the inner periphery 2021 of the fallopian tube 202 and the first protruding part 211A of the removing unit 21 touches the inner periphery 2021, the balloon 20 is moved backward to cause the first protruding part 211A to scrap off the tissue in the inner periphery 2021 of the fallopian tube 202 near the lesion 204 (a site in the proximal direction of the lesion 204). After that, in the extraction step, the catheter system 10 is extracted from the uterine fundus 200.

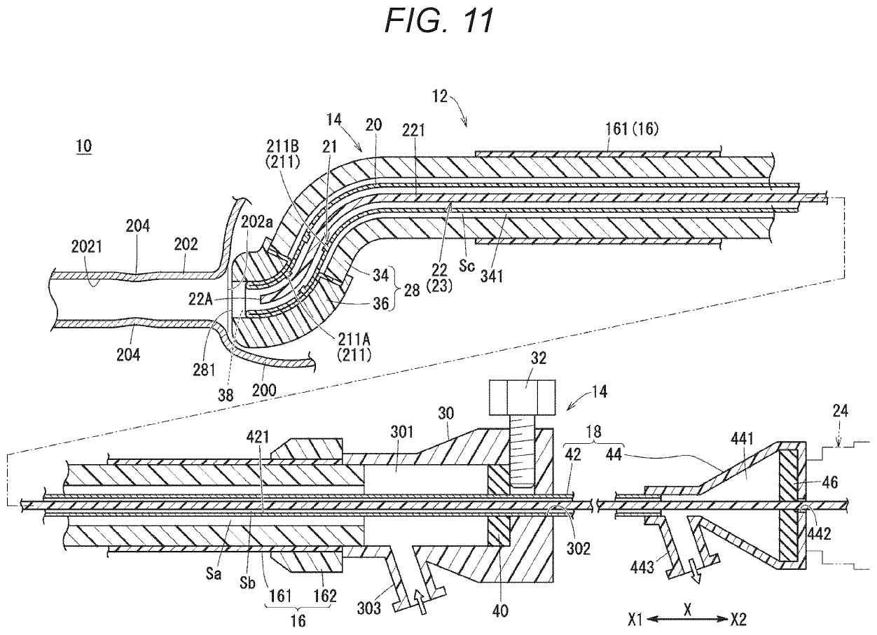

The tissue in the living body removed by the catheter system 10 is not limitedly collected after the extraction step. In falloposcopic tuboplasty according to a second modification illustrated in FIG. 11, after the lesion 204 is expanded radially outward by the balloon 20 in the forward movement step, the supply of the perfusate from the second feeding port 443 to the inner lumen Sb is stopped. A suction device (syringe or the like) (not illustrated) is connected to the second feeding port 443, and the suction device sucks the perfusate in the inner lumen Sb and the second space 441, whereby the tissue in the living body is taken out from the second feeding port 443 through the second space 441 of the inner tube hub 44 together with the perfusate supplied to the distal side of the balloon 20.

The first embodiment has the following effects.

As illustrated in FIG. 2, the catheter system 10 includes the outer tube 14, the inner tube 18 movably disposed in the first inner cavity 341 of the outer tube 14, the tubular balloon 20 connecting the distal portion of the outer tube 14 and the distal portion of the inner tube 18 to each other, and the removing unit 21 protruding from the distal end of the outer tube 14 in the distal direction (direction of arrow X1) and inserted into the fallopian tube 202 (lumen) of a living body to remove tissue in the fallopian tube 202. As illustrated in FIG. 8, the removing unit 21 is formed in the outer peripheral portion 203 of the balloon 20 in contact with the inner periphery 2021 of the fallopian tube 202.

Accordingly, when the balloon 20 travels to the fallopian tube 202 of the living body along the fallopian tube 202, it is possible to remove the tissue in the fallopian tube 202 effectively by the removing unit 21.

The removing unit 21 is the protrusion 211 (or recess) formed in the outer peripheral portion 203 of the balloon 20. Accordingly, the balloon 20 is provided with the removing unit 21 as the protrusion 211 (or the recess), thereby enabling removal of the tissue in the fallopian tube 202 with the balloon 20 and also enabling a simple structure of the catheter system 10 and reduction in manufacturing cost as compared with a configuration in which the removing unit 21 is designed as a separate member. Providing the balloon 20 with the protrusion 211 (or the recess) easily achieves the removing unit 21.

The inner tube 18 has a proximal end provided with the inner tube hub 44 having the second feeding port 443 capable of supplying and discharging a perfusate, and the second feeding port 443 is communicated with the second inner cavity 421 of the inner tube 18 and the inner cavity 201 of the balloon 20. Accordingly, as illustrated in FIG. 11, when the tissue is removed by the removing unit 21 of the balloon 20 in a state in which the perfusate is supplied to the balloon 20 through the second feeding port 443, discharging the perfusate from the second feeding port 443 makes it possible to take out and effectively collect the tissue from the second feeding port 443 together with the perfusate.

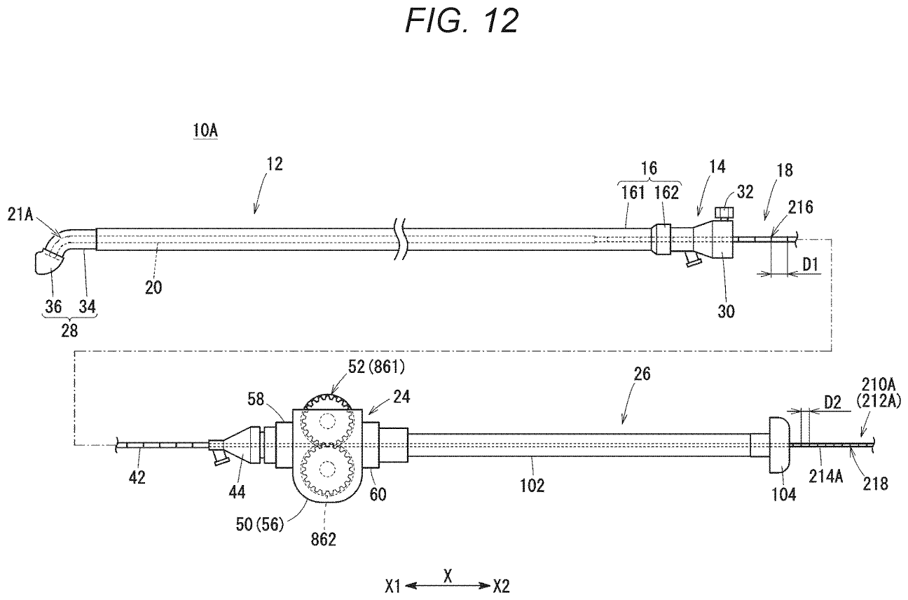

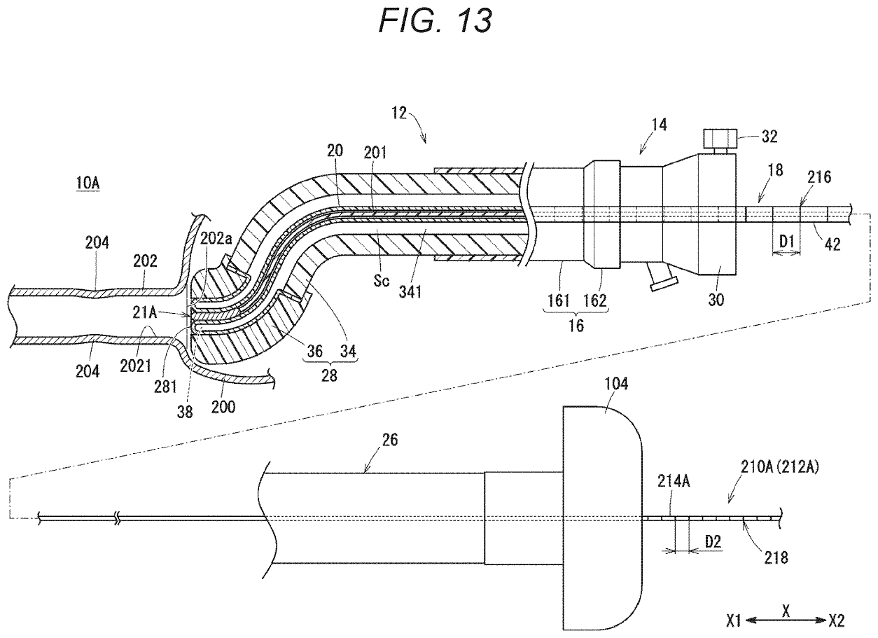

As illustrated in FIG. 12, a catheter system 10A according to a second embodiment includes a removing unit 21A protruding in the distal direction from a distal end of an outer tube 14 and inserted into a fallopian tube 202 of a living body to remove tissue in the fallopian tube 202. As illustrated in FIG. 13, the removing unit 21A is a part of a rod-like member 210A inserted into a balloon 20. The rod-like member 210A is composed of, for example, a cotton swab 212A. The rod-like member 210A has a body section 214A, and the body section 214A has a distal portion provided with the removing unit 21A. The rod-like member 210A viewed from an extending direction has a maximum diameter equal to or smaller than that of an inner cavity 201 of the balloon 20.

As illustrated in FIG. 13, the catheter system 10A includes first scale marks 216 disposed in an inner tube 18 and second scale marks 218 disposed in the body section 214A of the rod-like member 210A. The first scale marks 216 are disposed in the outer periphery of an inner tube main body 42. The first scale marks 216 are disposed at regular intervals in the axial direction (directions of arrow X) of the inner tube main body 42. The second scale marks 218 are formed on the outer periphery of the body section 214A in the rod-like member 210A. In the axial direction of the rod-like member 210A, an interval D2 between the second scale marks 218 is half the length of an interval D1 between the first scale marks 216.

Hereinafter described is a case in which tissue in a living body is removed by the catheter system 10A. First, in insertion step, an endoscope 22 is moved forward into the fallopian tube 202 of the living body to check a uterine ostium 202a with an image captured by the endoscope 22, and a lesion 204 is pushed and spread out by an outer peripheral portion 203 of the balloon 20.

Instead of checking the uterine ostium 202a with the endoscope 22, for example, the uterine ostium 202a may be checked with a hysteroscope without the endoscope 22. Alternatively, the uterine ostium 202a may be checked from the outside of the fallopian tube 202 with a laparoscope or X-rays.

Next, after the endoscope 22 is extracted from the balloon 20 and the inner tube 18 in the proximal direction (direction of arrow X2), the rod-like member 210A is inserted into the inner cavity 201 of the balloon 20 through a second inner cavity 421 of the inner tube 18, and the rod-like member 210A is moved in the distal direction relative to the balloon 20. As illustrated in FIG. 13, in the axial direction of the catheter system 10A, the distal end of the outer tube 14 (a distal end of a distal member 36), a distal end of the balloon 20, and a distal end of the rod-like member 210A are aligned. The distal end of the balloon 20 is inserted into the fallopian tube 202 of the living body. With respect to the balloon 20 and the inner tube 18, the rod-like member 210A is relatively moved in the proximal direction (direction of arrow X2) by one second scale mark 218, and then, the inner tube 18 is relatively moved in the distal direction (direction of arrow X1) by one first scale mark 216. The balloon 20 is folded back. For this reason, a moving distance of the balloon 20 in the distal direction is twice the moving distance of the rod-like member 210A in the axial direction. Therefore, a moving distance of the balloon 20 in the distal direction when the inner tube 18 is moved in the distal direction by one second scale mark 218 is equal to a moving distance of the rod-like member 210A when the rod-like member 210A is moved in the proximal direction by one first scale mark 216.

The distal end of the rod-like member 210A does not protrude in the distal direction (direction of arrow X1) from the distal end of the balloon 20 by moving the rod-like member 210A in advance in the proximal direction relative to the inner tube 18 and the balloon 20. That is to say, the distal end of the rod-like member 210A moves in the distal direction in a state in which the rod-like member 210A is covered by the balloon 20 without protruding from the distal end of the balloon 20. In other words, the rod-like member 210A is prevented from protruding from the balloon 20 in the distal direction before the distal end of the balloon 20 reaches the lesion 204, or a target position.

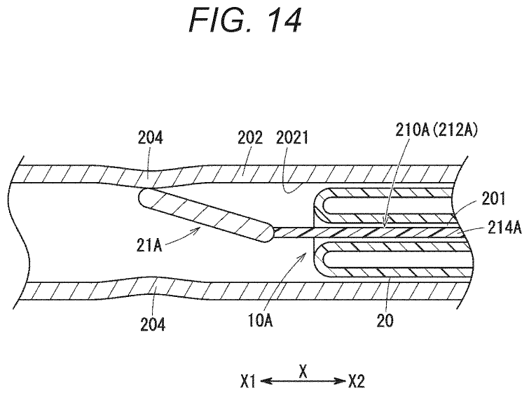

As illustrated in FIG. 14, after the distal end of the balloon 20 reaches a position facing the lesion 204, the rod-like member 210A is moved in the distal direction relative to the inner tube 18. The removing unit 21A disposed in a distal portion of the rod-like member 210A protrudes in the distal direction from the distal end of the balloon 20. The distal portion of the rod-like member 210A is tilted toward inner periphery 2021 of the fallopian tube 202, and the removing unit 21A is brought into contact with the inner periphery 2021 near the lesion 204, whereby tissue in the lesion 204 adheres to the surface of the removing unit 21A. The tissue in the inner periphery 2021 of the fallopian tube 202 is removed by the removing unit 21A. In the insertion step, note that a moving distance (insertion length) of the inner tube 18 in the distal direction is substantially equal to a moving distance (insertion length) of the endoscope 22 when the uterine ostium 202a is checked by the endoscope 22 before the insertion step.

After the tissue in the living body is removed by the removing unit 21A, the rod-like member 210A is moved in the proximal direction (direction of arrow X2) relative to the balloon 20 and taken out from a proximal end of the inner tube 18. Note that the present invention is not limited to the case in which the rod-like member 210A is taken out prior to the balloon 20. For example, the rod-like member 210A and the balloon 20 may be moved together in the proximal direction and extracted from uterine fundus 200 of the living body.

The second embodiment has the following effects.

As illustrated in FIG. 13, the catheter system 10A includes the outer tube 14, the inner tube 18 movably disposed in a first inner cavity 341 of the outer tube 14, the tubular balloon 20 connecting a distal portion of the outer tube 14 and a distal portion of the inner tube 18 to each other, and the removing unit 21A protruding from the distal end of the outer tube 14 in the distal direction and inserted into the fallopian tube 202 (lumen) of a living body to remove tissue in the fallopian tube 202. The removing unit 21A is a part of the rod-like member 210A inserted into the inner cavity 201 of the balloon 20.

Accordingly, when the balloon 20 travels into the fallopian tube 202 of the living body, it is possible to remove the tissue in the fallopian tube 202 by the removing unit 21A.

The removing unit 21A is disposed in the distal portion of the rod-like member 210A. Accordingly, the distal portion of the rod-like member 210A is provided with the removing unit 21A, enabling removal of tissue in the fallopian tube 202 more and reliably.

The inner tube 18 has the first scale marks 216 disposed at regular intervals in the axial direction, and the rod-like member 210A has the second scale marks 218 which are disposed at regular intervals in the axial direction from the distal end toward the proximal end and are half the length of each interval of the first scale marks 216 in the axial direction. With this configuration, when inserting the distal end of the balloon 20 into the fallopian tube 202 of the living body, the rod-like member 210A is relatively moved in the proximal direction based on the second scale marks 218, and then, the inner tube 18 is moved in the distal direction based on the first scale marks 216, thereby preventing the rod-like member 210A from protruding in the distal direction from the balloon 20 before the distal end of the balloon 20 reaches a target position, or the lesion 204, in the fallopian tube 202.

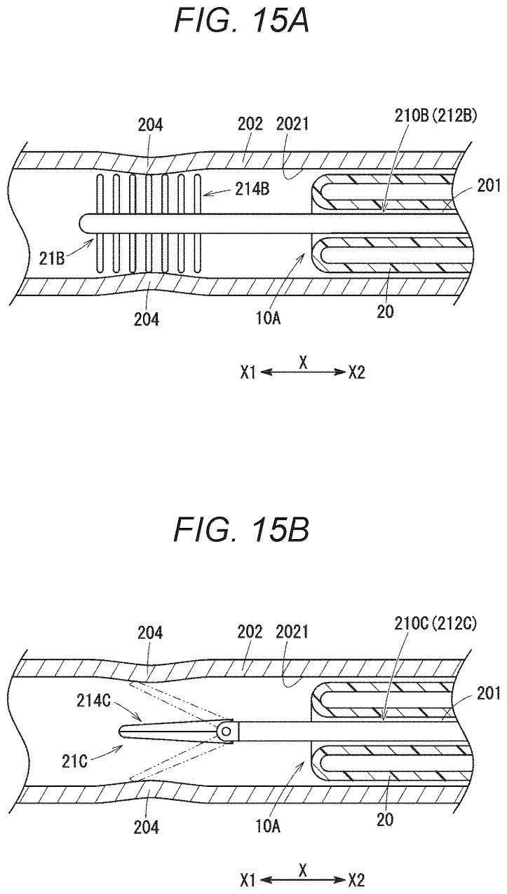

Note that the rod-like member 210A is not limited to the cotton swab 212A having a distal portion provided with the removing unit 21A. As illustrated in FIG. 15A, a rod-like member 210B may be a brushing member 212B having a distal end provided with a removing unit 21B. A distal portion of the brushing member 212B includes a brush 214B. The brush 214B is the removing unit 21B. The tissue in the fallopian tube 202 of the living body can be removed by the brush 214B. As illustrated in FIG. 15B, a rod-like member 210C may be a forceps member 212C having a distal end provided with a removing unit 21C. The forceps member 212C has a distal portion provided with forceps 214C. The forceps 214C can be opened and closed, and the tissue in the fallopian tube 202 of the living body can be removed by closing the forceps 214C which is in the open state.

Note that the present invention is not limited to the disclosure, and various configurations can be adopted without departing from the gist of the present invention.

What is claimed is:

1. A catheter system configured to be inserted into a lumen of a living body and to travel along the lumen, the catheter system comprising:
a flexible outer tube;
an inner tube disposed in an inner cavity of the outer tube so as to be movable in an axial direction of the outer tube;
a balloon having a tubular shape that connects a distal portion of the outer tube and a distal portion of the inner tube to each other, the balloon being configured to inflate radially inward with respect to the outer tube to allow insertion of a rod-like member through the inner tube, wherein the balloon has an outer peripheral portion that touches inner periphery of the lumen when the balloon protrudes in the distal direction from the distal end of the outer tube; and
a removing unit protruding in a distal direction from a distal end of the outer tube and inserted into the lumen of the living body to remove tissue in the lumen, wherein the removing unit is (i) a protrusion or recess formed at the outer peripheral portion of the balloon, or (ii) a part of the rod-like member inserted into the balloon.

2. The catheter system according to claim 1, wherein:
the removing unit is the protrusion or the recess formed at the outer peripheral portion of the balloon.

3. The catheter system according to claim 2, wherein:
the inner tube comprises an inner tube hub at a proximal end of the inner tube, the inner tube hub comprising a port capable of supplying and discharging a perfusate, and
the port communicates with an inner part of the inner tube and an inner part of the balloon.

4. The catheter system according to claim 1, further comprising:
the rod-like member, wherein:
the removing unit is disposed in a distal portion of the rod-like member.

5. The catheter system according to claim 4, wherein:
the inner tube has a first scale mark disposed at a regular interval in the axial direction, and
the rod-like member has a second scale mark that is disposed at a regular interval in the axial direction from a distal end toward a proximal end and is half a length of the interval of the first scale mark in the axial direction.

6. The catheter system according to claim 1, wherein:
the rod-like member is an endoscope.

7. A catheter system configured to be inserted into a lumen of a living body and to travel along the lumen, the catheter system comprising:
a flexible outer tube;
an inner tube disposed in an inner cavity of the outer tube so as to be movable in an axial direction of the outer tube; and
a balloon having a tubular shape that connects a distal portion of the outer tube and a distal portion of the inner tube to each other, the balloon being configured to inflate radially inward with respect to the outer tube to allow insertion of a rod-like member through the inner tube, wherein the balloon has an outer peripheral portion that touches inner periphery of the lumen when the balloon protrudes in the distal direction from the distal end of the outer tube;
wherein the balloon comprises a protrusion or recess located at the outer peripheral portion of the balloon and configured to remove tissue in the lumen.

8. The catheter system according to claim 7, wherein:
the inner tube comprises an inner tube hub at a proximal end of the inner tube, the inner tube hub comprising a port capable of supplying and discharging a perfusate, and
the port communicates with an inner part of the inner tube and an inner part of the balloon.

9. The catheter system according to claim 7, wherein:
the rod-like member is an endoscope.

10. A catheter system configured to be inserted into a lumen of a living body and to travel along the lumen, the catheter system comprising:
a flexible outer tube;
an inner tube disposed in an inner cavity of the outer tube so as to be movable in an axial direction of the outer tube;
a rod-like member comprising a swab; and
a balloon having a tubular shape that connects a distal portion of the outer tube and a distal portion of the inner tube to each other, the balloon being configured to inflate radially inward with respect to the outer tube to allow insertion of the rod-like member through the inner tube, wherein the balloon has an outer peripheral portion that touches inner periphery of the lumen when the balloon protrudes in the distal direction from the distal end of the outer tube.

11. The catheter system according to claim 10, wherein:
the inner tube has a first scale mark disposed at a regular interval in the axial direction, and
the rod-like member has a second scale mark that is disposed at a regular interval in the axial direction from a distal end toward a proximal end and is half a length of the interval of the first scale mark in the axial direction.

12. The catheter system according to claim 10, wherein:
the rod-like member is a cotton swab.
